(19) United States
(12) Patent Application Publication
(10) Pub. No.: US 2023/0366037 A1
(43) Pub. Date: Nov. 16, 2023

(54) PREDICTION TOOL FOR JUDGING DRUG SENSITIVITY AND LONG-TERM PROGNOSIS OF LIVER CANCER BASED ON GENE DETECTION AND USE THEREOF

(71) Applicant: ZHEJIANG UNIVERSITY, Hangzhou (CN)

(72) Inventors: Junjie XU, Hangzhou (CN); Xiujun CAI, Hangzhou (CN); Yu PAN, Hangzhou (CN); Xiao LIANG, Hangzhou (CN); Shunjie XIA, Hangzhou (CN)

(21) Appl. No.: 18/356,261

(22) Filed: Jul. 21, 2023

Related U.S. Application Data
(63) Continuation of application No. PCT/CN2022/072196, filed on Jan. 15, 2022.

(30) Foreign Application Priority Data
Jan. 21, 2021 (CN) 202110080948.2

(51) Int. Cl. C12Q 1/6886

(52) U.S. Cl. CPC C12Q 1/6886; C12Q 2600/106; C12Q 2600/118; C12Q 2600/158

(57) ABSTRACT

A prediction tool for judging drug sensitivity and long-term prognosis of liver cancer based on gene detection provided. The present application statistically analyzes an aerobic glycolysis pathway gene related to liver cancer prognosis in TCGA data, and adopts LASSO regression analysis to simplify a prognosis-related gene on this basis, so as to establish a prediction tool based on the aerobic glycolysis pathway gene, referred to as an aerobic glycolysis index. The index is validated in a plurality of public databases and clinical samples from a Sir Run Run Shaw Hospital, and it is found that the index can accurately predict sensitivity and long-term prognosis of liver cancer patients to sorafenib therapy. The present application can effectively screen liver cancer patients sensitive to the sorafenib therapy, and provides a new idea for precise and comprehensive treatment of the liver cancer patients.

### CROSS-REFERENCE TO RELATED APPLICATIONS

The present application is a National Stage of International Application No. PCT/CN2022/072196, filed on Jan. 15, 2022, which claims priority to Chinese Patent Application No. 202110080948.2, filed on Jan. 21, 2021, both of which are hereby incorporated by reference in their entireties.

### TECHNICAL FIELD

The present application belongs to the fields of biotechnology and medicine, and particularly relates to gene detection related to anti-tumor drug resistance and use thereof.

### BACKGROUND

Liver cancer is the sixth most common malignant tumor in our country and around the world, and meanwhile ranks fourth among tumor-related causes of death. Although great progress has been made in treatment methods, a five-year survival rate for liver cancer is still between 25% and 55%. Distant metastasis, intrahepatic recurrence and low sensitivity to various treatment methods are main reasons for poor prognosis of the liver cancer. Gene mutation, chromosome abnormality, and abnormal cell signaling pathway are closely related to occurrence and development of the liver cancer. Typing of the liver cancer by molecular biological characteristics can help achieve precise treatment and improve prognosis of liver cancer patients.

Aerobic glycolysis is a hallmark feature of tumor malignancy, which mainly means that even when an oxygen concentration is at a physiological concentration, tumor cells still obtain a large amount of energy through glycolysis. Through this change in glucose metabolism, tumor cells can further produce a large number of metabolic products required for physiological synthesis while quickly obtaining energy. In addition, aerobic glycolysis is closely related to a variety of oncogene signaling pathways. Therefore, predicting the liver cancer by a level of aerobic glycolysis may reveal new molecular typing of liver cancer.

Sorafenib is currently a first-line therapeutic drug for advanced liver cancer. But a sorafenib resistance phenomenon is very common in clinic. How to screen out patients who are sensitive to sorafenib therapy and precise drug use are crucial to improving prognosis of the liver cancer patients. Tumor metabolism, tumor microenvironment change, and epigenetics and the like are also considered to be possibly related to sorafenib resistance in liver cancer. However, a dominant mechanism or a key gene is still the main problem that plagues the research of sorafenib resistance in liver cancer at present.

Therefore, there is an urgent need in the field to find a new method that can predict the sensitivity of the liver cancer to sorafenib and the long-term prognosis of the liver cancer, so as to achieve precise treatment and improve the prognosis of the patients.

### SUMMARY

The objective of the present application is to find a new prediction tool for predicting sensitivity and long-term prognosis of liver cancer to sorafenib against deficiencies of the prior art.

The present application is achieved through the following technical solutions:
1. an aerobic glycolysis pathway gene related to liver cancer prognosis in TCGA data is statistically screened by using an univariate Cox regression model; and
2. LASSO regression analysis is adopted to simplify a prognosis-related gene on this basis, and a prediction tool based on the aerobic glycolysis pathway gene, referred to as an aerobic glycolysis index, is established, wherein the aerobic glycolysis index=LDHA gene expression level*0.163+STC2 gene expression level*0.004+GPC1 gene expression level*0.034+TKTL1 gene expression level*0.0001+SLC2A1 gene expression level*0.014+SRD5A3 gene expression level*0.032+PLOD2 gene expression level*0.070+G6PD gene expression level*0.083+HMMR gene expression level*0.040+HOMER1 gene expression level*0.001+RARS1 gene expression level*0.132-GOT2 gene expression level*0.146+CENPA gene expression level*0.053-SLC2A2 gene expression level*0.001.

A detection method/technology of the gene expression level includes: a second-generation RNA sequencing or third-generation RNA sequencing or gene chip technology.
3. The index is verified in a plurality of public databases and clinical samples from a Sir Run Run Shaw Hospital, and it is found that the index can accurately predict the long-term prognosis of a liver cancer patient; and a “survminer” data package is used to obtain an optimal threshold of the aerobic glycolysis index corresponding to a corresponding detection method of a data set according to survival data of the data set, if the aerobic glycolysis index of the patient is higher than the threshold, it indicates that the prognosis of the liver cancer patient is poor, otherwise, it indicates that the prognosis of the liver cancer patient is good.
4. The index is validated in GDSC and CCLE databases and clinical samples of an “STORM” experiment, and it is found that the index is negatively correlated with sorafenib sensitivity and can accurately predict the sensitivity of the patient to sorafenib therapy. The “survminer” data package is used to obtain the optimal threshold of the aerobic glycolysis index corresponding to the corresponding detection method of the data set according to the survival data of the data set, if the aerobic glycolysis index of the patient is higher than the threshold, it indicates that the patient has poor sensitivity to the sorafenib therapy, otherwise, it indicates that the patient has good sensitivity to the sorafenib therapy.

The present application further provides a kit for judging drug sensitivity and long-term prognosis of liver cancer based on gene detection, containing a reagent for measuring expression levels of an LDHA gene, an STC2 gene, a GPC1 gene, a TKTL1 gene, an SLC2A1 gene, an SRD5A3 gene, a PLOD2 gene, a G6PD gene, an HMMR gene, an HOMER1 gene, a RARS1 gene, a GOT2 gene, a CENPA gene and an SLC2A2 gene.

Preferably, the reagent is a primer or probe that specifically binds to the gene.

The present application has the beneficial effects that: the index of the present application is only based on the expression levels of 14 genes, the method is simple, the prediction accuracy is high, promotion is easy, and it has very good clinical transformation value.

### BRIEF DESCRIPTION OF DRAWINGS

The present application is further illustrated below in conjunction with accompanying drawings and embodiments.

FIG. 1 shows that 80 aerobic glycolysis-related genes are associated with prognosis of liver cancer prompted by univariate Cox analysis.

FIG. 2 shows that prognosis-related genes are simplified by LASSO regression analysis, and an aerobic glycolysis index based on expression levels of 14 genes is established.

FIG. 3 is a curve diagram showing that an aerobic glycolysis index can predict an overall survival rate (a) and a disease-free survival rate (b) of liver cancer patients in a TCGA database; and in the figure, 2 represents a survival curve of low AGI, and 1 and 3 are respectively error bars of the survival curve of low AGI; and 5 represents a survival curve of high AGI, and 4 and 6 are respectively error bars of the survival curve of high AGI.

FIG. 4 is an ROC curve diagram of TCGA-LIHC data.

FIG. 5 is a curve diagram showing that an aerobic glycolysis index can predict an overall survival rate of liver cancer patients (c) in GSE14520 (a) and LIRI-JP databases (b) and a Sir Run Run Run Shaw hospital; and in the figure, 2 represents a survival curve of low AGI, and 1 and 3 are respectively error bars of the survival curve of low AGI; and 5 represents a survival curve of high AGI, and 4 and 6 are respectively error bars of the survival curve of high AGI.

FIG. 6 is a curve diagram showing negative correlation between sensitivity of a liver cancer cell line in GDSC (a) and CCLE databases (b) to sorafenib and an aerobic glycolysis index.

FIG. 7 is “STORM” clinical data showing that an aerobic glycolysis index can predict response to sorafenib therapy.

FIG. 8 is an AUC curve diagram of “STORM” clinical data.

### DESCRIPTION OF EMBODIMENTS

The present application will be further illustrated below through experiments and in conjunction with embodiments. It should be understood that these embodiments are only used for the purpose of example illustration, and in no way limit the protection scope of the present application.

Sources of sequencing and clinical data and reagents involved in the present embodiment:

TCGA-LIHC data are downloaded from a UCSC database (https://xenabrowser.net/datapages), and LIRI-JP data are downloaded from an HCCDB database (http://lifeome.net/database/hccdb/download.html). GSE14520 and GSE109211 data are downloaded from a GEO database (https://www.ncbi.nlm.nih.gov/geo/). Data of sensitivity of liver cancer cell lines to sorafenib are downloaded from a GDSC database (https://www.cancerrxgene.org) and a CCLE database (https://portals.broadinstitute.org/ccle/data). Data from a Sir Run Run Shaw Hospital is derived from 102 cases of medical treatment in the Sir Run Run Shaw Hospital affiliated to Zhejiang University School of Medicine from January 2008 to January 2018. Among the 102 cases of medical treatment, all were diagnosed with liver cancer, TNM staging was I-IV, T staging was T1-T4, age was 32-88 years old, and the clinical follow-up time was more than 2 years.

### Embodiment

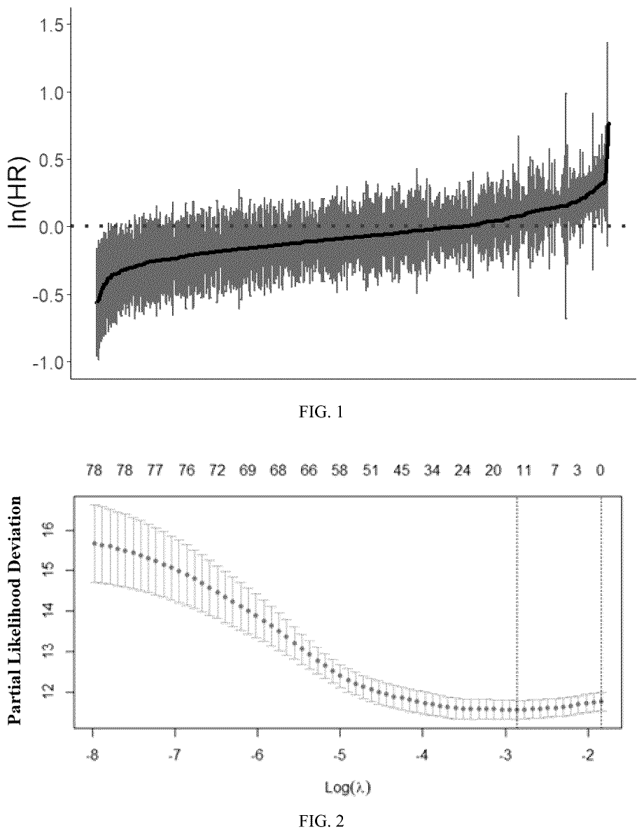

Sequencing data and clinical follow-up information of 371 liver cancer patients in the TCGA-LIHC data are selected, and influence of an aerobic glycolysis gene on an overall survival rate of these 371 patients is analyzed by univariate COX regression. Results show that a total of 80 genes significantly influence the overall survival rate of the liver cancer patients, as shown in FIG. 1.

A prognosis-related gene will be simplified through LASSO regression analysis, an aerobic glycolysis index based on expression levels of 14 genes is established, as shown in FIG. 2, and assignment is specifically: aerobic glycolysis index (AGI)=LDHA gene expression*0.163 wherein the aerobic glycolysis index=LDHA gene expression level*0.163+STC2 gene expression level*0.004+GPC1 gene expression level*0.034+TKTL1 gene expression level*0.0001+SLC2A1 gene expression level*0.014+SRD5A3 gene expression level*0.032+PLOD2 gene expression level*0.070+G6PD gene expression level*0.083+HMMR gene expression level*0.040+HOMER1 gene expression level*0.001+RARS1 gene expression level*0.132-GOT2 gene expression level*0.146+CENPA gene expression level*0.053-SLC2A2 gene expression level*0.001.

Cases can be grouped based on the aerobic glycolysis index (AGI), wherein a threshold for grouping is a point where prognosis of the two groups of patients differs the most. For example, an optimal threshold is obtained by using a R language software “survminer” data package according to survival data of the patients. It should be pointed out that the threshold will be different for different sequencing methods. The following is a detailed description in conjunction with a specific validation set:

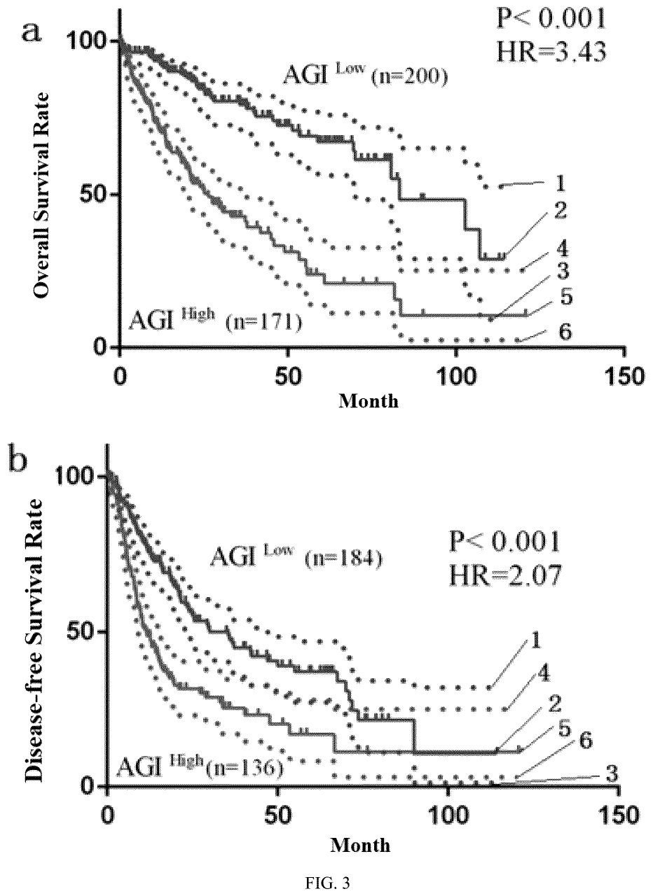

the influence of the aerobic glycolysis index on long-term prognosis of the liver cancer patients is verified in TCGA-LIHC data, that is, an Illumina HiSeq 2000 RNA sequencing platform is used to detect the expression level of each gene in liver cancer tissue of the patients. After standardization processing, the aerobic glycolysis index of each liver cancer patient is calculated. According to the patient survival data, the R language software “survminer” data package is used to take the optimal threshold of 4.05. The aerobic glycolysis index being lower than 4.05 is a low aerobic glycolysis index group (low AGI group), and the aerobic glycolysis index being higher than 4.05 is a high aerobic glycolysis index group (high AGI group). Through Kaplan-Meier survival curve and log-rank survival analysis, it is found that the aerobic glycolysis index indicates that the liver cancer patients in the high AGI group have a worse long-term prognosis, including overall survival rate and disease-free survival rate, as shown in FIG. 3.

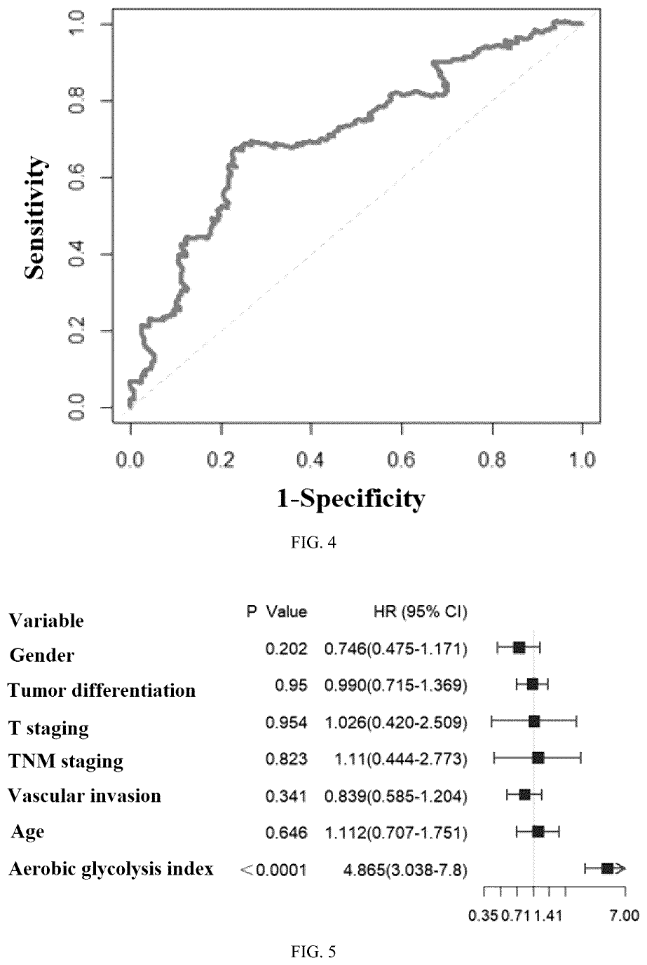

At the same time, the ROC curve diagram is applied to evaluate clinical accuracy of a model in the present embodiment. The ROC curve is shown in FIG. 4. Abscissa is 1-specificity, ordinate is the sensitivity, in a case that the five-year survival rate is a node, when 4.05 is taken, its specificity is 0.65, the sensitivity is 0.69, and an AUC value of a calculation model is 0.714, indicating that the model prediction result is high in accuracy. An area under the ROC curve is between 1.0 and 0.5. When the AUC is greater than 0.5, the closer the AUC is to 1, the better the diagnostic effect.

Further, COX regression analysis is adopted to verify related risk factors of the aerobic glycolysis index on the long-term prognosis of the liver cancer patients in TCGA. Multivariate regression analysis found that clinical indexes such as age (greater than or equal to 60 years old, control is less than 60 years old), gender (male, control is female), tumor differentiation (G3 grade, G2 grade, control is G1 grade), tumor stage (stage IV, stage III, stage II, control is stage I), vascular invasion (macro-vascular invasion, micro-invasion, and control is no invasion) are not independent risk factors for the long-term prognosis of the liver cancer patients, and the aerobic glycolysis index is the independent risk factor for the long-term prognosis of the liver cancer patients, as shown in FIG. 5. The results indicate that the aerobic glycolysis index of the present application can be utilized to independently predict the long-term prognosis of the liver cancer patients, without being influenced by the clinical indicators such as the age, the gender, the tumor differentiation, the tumor stage, and the vascular invasion.

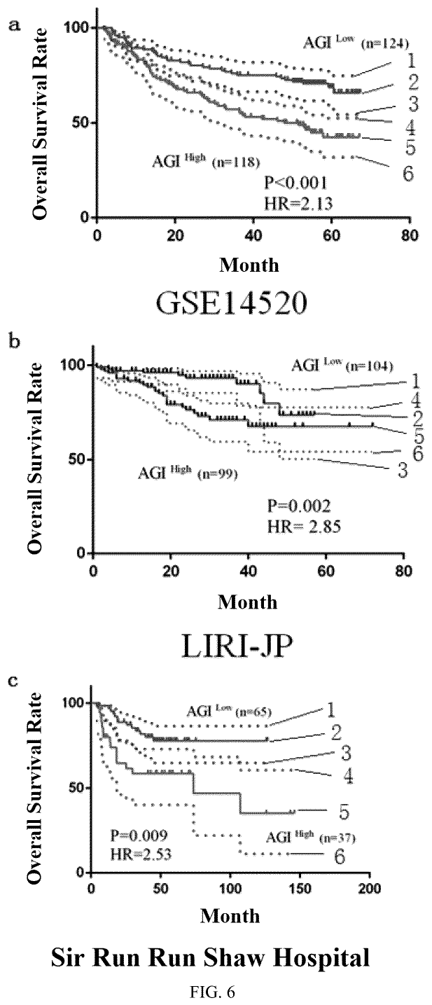

The influence of the aerobic glycolysis index on the long-term prognosis of the liver cancer patients is further verified in 243 liver cancer patients in the GSE14520 database, 200 liver cancer patients in the LIRI-JP database, and 102 liver cancer patients in the Sir Run Run Shaw Hospital. Similarly, Affymetrix Human Genome U133A 2.0 Array (GSE14520), Illumina RNA-Seq (LIRI-JP), and Illumina (Sir Run Run Shaw Hospital) sequencing platforms are used to detect the expression level of each gene in the liver cancer tissue of the patients. After standardization, the aerobic glycolysis index of each liver cancer patient is calculated and the optimal threshold is taken (3.245 (GSE14520), 1.785 (LIRI-JP), 1.64 (Sir Run Run Shaw Hospital)). The aerobic glycolysis index lower than the optimal threshold is the low AGI group, and the AGI higher than the optimal threshold is the high AGI group. The aerobic glycolysis index prompts a worse overall survival rate of the liver cancer patients in the high AGI group, as shown in FIG. 6.

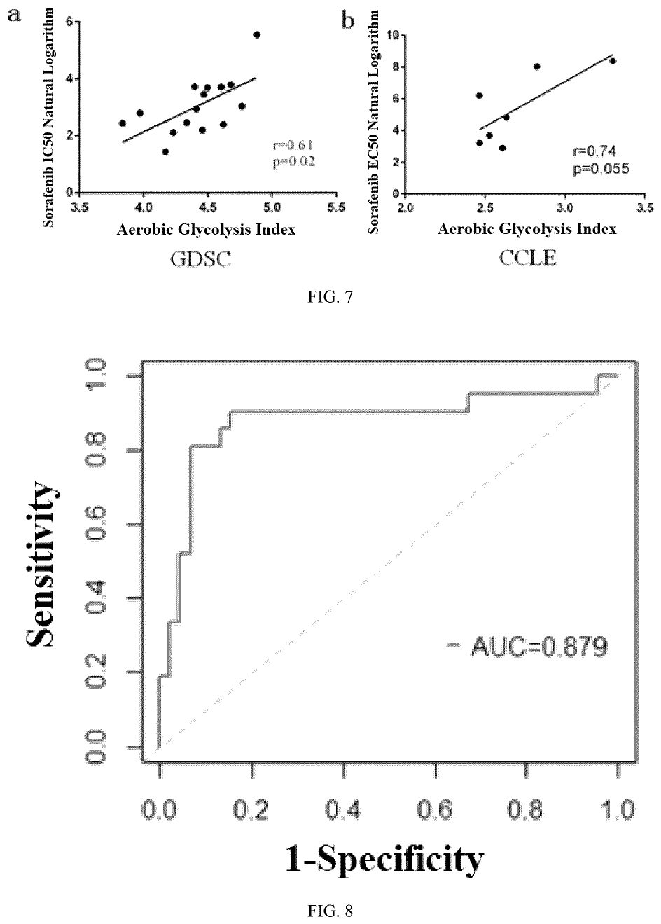

In the GDSC database, it is suggested that an IC50 concentration of the liver cancer cell line to sorafenib is positively correlated with the aerobic glycolysis index. In the CCLE database, it is suggested that an EC50 concentration of the liver cancer cell line to sorafenib is positively correlated with the aerobic glycolysis index, as shown in FIG. 7a and FIG. 7b.

In 67 liver cancer patients treated with sorafenib in the “STORM” database, the aerobic glycolysis index can effectively predict the sensitivity of the liver cancer patients to sorafenib, and the area under the curve is 0.879, as shown in FIG. 8. A threshold of 3.488 corresponds to a sensitivity of 0.905 and a specificity of 0.848.

The present embodiment further provides a method for predicting sensitivity of a patient to sorafenib therapy by using the present application, which specifically includes the following steps:
1. tissue samples (such as surgical specimens and puncture specimens) of liver cancer patients are collected, and total RNA in the tissue is extracted.
2. A suitable sequencing platform is selected to detect a gene related to an aerobic glycolysis index and calculate the aerobic glycolysis index.
3. According to an established aerobic glycolysis index database, an aerobic glycolysis index level of the samples is judged by referring to an optimal threshold of the aerobic glycolysis index in the database.
4. If the aerobic glycolysis index level of the detected samples is lower than the threshold, the patient has good prognosis and is sensitive to sorafenib therapy, and sorafenib adjuvant therapy can be performed to improve prognosis. Otherwise, if the aerobic glycolysis index level of the detected samples is higher than the threshold, the patient has poor prognosis, is not sensitive to the sorafenib therapy, and is not suitable for being subjected to sorafenib adjuvant therapy.

What is claimed is:

1. Use of a gene testing reagent in preparation of a kit for judging sensitivity of a patient to liver cancer drug Sorafenib, wherein the gene testing reagent is a reagent for measuring expression levels of an LDHA gene, an STC2 gene, a GPC1 gene, a TKTL1 gene, an SLC2A1 gene, an SRD5A3 gene, a PLOD2 gene, a G6PD gene, an HMMR gene, an HOMER1 gene, a RARS1 gene, a GOT2 gene, a CENPA gene, and an SLC2A2 gene.

2. The use according to claim 1, wherein the reagent is a primer or a probe that specifically binds to the gene.

3. Use of a gene testing reagent in preparation of a kit for predicting long-term prognosis of a liver cancer patient, wherein the gene testing reagent is a reagent for measuring expression levels of an LDHA gene, an STC2 gene, a GPC1 gene, a TKTL1 gene, an SLC2A1 gene, an SRD5A3 gene, a PLOD2 gene, a G6PD gene, an HMMR gene, an HOMER1 gene, a RARS1 gene, a GOT2 gene, a CENPA gene, and an SLC2A2 gene.

4. The use according to claim 3, wherein the reagent is a primer or probe that specifically binds to the gene.

5. A method for constructing a prediction tool for predicting sensitivity and long-term prognosis of a liver cancer patient to Sorafenib, specifically comprising:
(1) statistically screening an aerobic glycolysis pathway gene related to liver cancer prognosis in TCGA data by using an univariate Cox regression model; and
(2) adopting LASSO regression analysis to simplify a prognosis-related gene on this basis, and establishing a prediction tool based on the aerobic glycolysis pathway gene, referred to as an aerobic glycolysis index, wherein the aerobic glycolysis index=LDHA gene expression level*0.163+STC2 gene expression level*0.004+GPC1 gene expression level*0.034+TKTL1 gene expression level*0.0001+SLC2A1 gene expression level*0.014+SRD5A3 gene expression level*0.032+PLOD2 gene expression level*0.070+G6PD gene expression level*0.083+HMMR gene expression level*0.040+HOMER1 gene expression level*0.001+RARS1 gene expression level*0.132-GOT2 gene expression level*0.146+CENPA gene expression level*0.053-SLC2A2 gene expression level*0.001,
wherein a detection technology of the gene expression level comprises: a second-generation RNA sequencing or third-generation RNA sequencing or gene chip technology.
